(19) United States
(12) Patent Application Publication
(10) Pub. No.: US 2007/0110812 A1
(43) Pub. Date: May 17, 2007

(54) Ophthalmic composition for dry eye therapy

(75) Inventors: Erning Xia, Penfield, NY (US); John Denick, Jefferson City, TN (US); Richard Smerbeck, Pittsford, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/595,384

(22) Filed: Nov. 9, 2006

Related U.S. Application Data
(60) Provisional application No. 60/736,522, filed on Nov. 14, 2005.

(51) Int. Cl. A61K 31/573; A61K 9/14

(52) U.S. Cl. 424/487; 514/179

(57) ABSTRACT

Disclosed in embodiments are gel formulations which are not subject to the settling out phenomena that may be observed with Loteprednol etabonate suspensions.

### CROSS REFERENCE TO RELATED APPLICATIONS

This patent application claims priority to US Provisional Patent Application No. 60/736,522 filed Nov. 14, 2005 and is incorporated herein by reference.

### BACKGROUND AND SUMMARY

Irritation and/or inflammation of the eye can arise from many causes. For example, it is known that seasonal allergic conjunctivitis can present signs and symptoms that include discomfort and ocular inflammation for the patent presenting with this condition. Other causes of eye irritation can arise from steroid responsive conditions of the palpebral bulbar conjunctiva, cornea and anterior segment of the globe. In addition, dry eye, also known generically as keratoconjunctivitis sicca (KCS), is a common ophthalmological disorder affecting millions of Americans each year. The condition is particularly widespread among post-menopausal women due to hormonal changes following the cessation of fertility. Dry eye may afflict an individual with varying severity. In mild cases, a patient may experience burning, a feeling of dryness, and persistent irritation such as is often caused by small bodies lodging between the eye lid and the eye surface. In severe cases, vision may be substantially impaired. Other diseases, such as Sjogren's disease manifest dry eye complications.

Corticosteroids are potent, non-specific anti-inflammatory drugs that inhibit a variety of chemotactic substances and factors that mediate capillary permeability, contraction of nonvascular smooth muscle, and vasodilatation. In addition, corticosteroids suppress inflammation by inhibiting edema, fibrin deposition, migration of leukocytes and phagocytic activity.

Topical corticosteroids are useful in a variety of ophthalmic conditions and are generally indicated for treatment of steroid-responsive inflammatory conditions of the palpebral and bulbar conjunctiva, cornea and anterior segment of the eye. Although corticosteroids are widely used as a topical agent for ocular inflammation, most possess a safety risk profile that limits their more general utility. A common risk associated with corticosteroid therapy is an elevation of intraocular pressure (IOP). In addition, chronic use of topical corticosteroids may result in the development of cataracts. Loteprednol etabonate is a compound designed as a site-active corticosteroid that will undergo a predictable transformation to an inactive metabolite. The relatively rapid metabolism of Loteprednol etabonate to an inactive metabolite improves the safety profile of this corticosteroid. This characteristic of Alrex® (Loteprednol etabonate ophthalmic suspension, 0.2%) and Lotemax® (Loteprednol etabonate ophthalmic suspension, 0.5%) makes them excellent candidates for use in inflammatory ocular conditions.

Although entirely satisfactory in there performance Alrex and Lotemax are suspensions that may result in an undesirable amount of settling of the active upon improper storage or inadequate shaking of the container before use of the drug product.

Therefore, disclosed in embodiments herein are gel formulations which are not subject to the settling out phenomena that may be observed with Loteprednol etabonate suspensions.

Also disclosed herein are pharmaceutically acceptable compositions comprising anionic polymers such as hyaluronic acid, alginates, carboxy methyl cellulose; water; osmotic agents such as propylene glycol, glycerin, sugars, mannitol, amino acid; chelating agent such as EDTA, DEQUEST; and any pharmaceutically active ingredient or combination of pharmaceutically active ingredients.

Further disclosed are methods for producing a sterile Loteprednol etabonate gel. The method is characterized in that Loteprednol etabonate is sterilized and incorporated into a sterile polyacrylate gel, which has been per se conventionally produced, in appropriate amount under aseptic conditions, or else the sterile Loteprednol etabonate or its pharmaceutically acceptable ester is suspended in a part of the amount of water required for producing the polyacrylate suspension and is then homogenously incorporated into the polyacrylate, which is then made into a gel.

### BRIEF DESCRIPTION OF THE FIGURES.

None.

### DETAILED DESCRIPTION

Topical steroids for treating ocular inflammations can be based on predictably metabolized drugs. Predictably metabolized drugs, as is known in the art, are designed to provide maximal therapeutic effect and minimal side effects. By one approach, synthesis of a “predictably metabolized drug” can be achieved by structurally modifying a known inactive metabolite of a known active drug to produce an active metabolite that undergoes a predictable one-step transformation in-vivo back to the parent, inactive metabolite (see, U.S. Pat. Nos. 6,610,675, 4,996,335 and 4,710,495 for predictably metabolized steroids). “Predictably metabolized drugs” therefore are biologically active chemical components characterized by predictable in-vivo metabolism to non-toxic derivatives after they provide their therapeutic effect. Formulations of steroids suitable for ophthalmic use are known. For example, U.S. Pat. Nos. 4,710,495, 4,996,335, 5,540,930, 5,747,061, 5,916,550, 6,368,616 and 6,610,675, the contents of each of which is incorporated by reference herein, describe predictably metabolized steroids and/or formulations containing predictably metabolized steroids.

(11β,17α),-17-[(Ethoxycarbonyl)oxy]-11-hydroxy-3-oxoandrosta-1,4-diene-17-carboxylic acid chloromethyl ester (Loteprednol etabonate) is a known compound and can be synthesized by methods disclosed in U.S. Pat. No. 4,996,335, the entire contents of which are hereby incorporated by reference in the present specification.

According to the methods of the present invention, a formulation comprising (11β,17α),-17-[(Ethoxycarbonyl)oxy]-11-hydroxy-3-oxoandrosta-1,4-diene-17-carboxylic acid chloromethyl ester and a pharmaceutically acceptable carrier for topical ophthalmic administration or implantation into the conjunctival sac or anterior chamber of the eye is administered to a mammal in need thereof. The formulations are formulated in accordance with methods known in the art for the particular route of administration desired.

The formulations administered according to the present invention comprise a pharmaceutically effective amount of (11β,17α),-17-[(Ethoxycarbonyl)oxy]-11-hydroxy-3-oxoandrosta-1,4-diene-17-carboxylic acid chloromethyl ester. As used herein, a “pharmaceutically effective amount” is one which is sufficient to reduce or eliminate signs or symptoms of dry eye. Generally, for formulations intended to be administered topically to the eye in the form of eye drops or eye ointments, the amount of (11β,17α),-17-[(Ethoxycarbonyl)oxy]-11-hydroxy-3-oxoandrosta-1,4-diene-17-carboxylic acid chloromethyl ester will be about 0.001 to 5.0% (W/W). For preferred topically administrable ophthalmic formulations, the amount of (11β,17α),-17-[(Ethoxycarbonyl)oxy]-11-hydroxy-3-oxoandrosta-1,4-diene-17-carboxylic acid chloromethyl ester will be about 0.001 to 1.0% (W/W).

The formulations administered according to the present invention may also include various other ingredients, including but not limited to surfactants, tonicity agents, buffers, preservatives, co-solvents and viscosity building agents.

Surfactants that can be used are surface-active agents that are acceptable for ophthalmic or otolaryngological uses. Useful surface active agents include but are not limited to polysorbate 80, tyloxapol, TWEEN 80 (ICI America Inc., Wilmington, Del.), PLURONIC F-68 (from BASF, Ludwigshafen, Germany) and the poloxamer surfactants can also be used. These surfactants are nonionic alkaline oxide condensates of an organic compound which contains hydroxyl groups. The concentration in which the surface active agent may be used is only limited by neutralization of the bactericidal effects on the accompanying preservatives (if present), or by concentrations which may cause irritation.

Various tonicity agents may be employed to adjust the tonicity of the formulation. For example, sodium chloride, potassium chloride, magnesium chloride, calcium chloride, nonionic diols, preferably glycerol, dextrose and/or mannitol may be added to the formulation to approximate physiological tonicity. Such an amount of tonicity agent will vary, depending on the particular agent to be added. In general, however, the formulations will have a tonicity agent in an amount sufficient to cause the final formulation to have an ophthalmically acceptable osmolality (generally about 150-450 mOsm).

An appropriate buffer system (e.g., sodium phosphate, sodium acetate, sodium citrate, sodium borate or boric acid) may be added to the formulations to prevent pH drift under storage conditions. The particular concentration will vary, depending on the agent employed.

Topical ophthalmic products are typically packaged in multidose form. Preservatives are thus required to prevent microbial contamination during use. Suitable preservatives include: biguanides, hydrogen peroxide, hydrogen peroxide producers, benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% W/W. Unit dose formulations of the present invention will be sterile, but typically unpreserved. Such formulations, therefore, generally will not contain preservatives.

Co-solvents and viscosity building agents may be added to the formulations to improve the characteristics of the formulations. Such materials can include nonionic water-soluble polymer. Other compounds designed to lubricate, “wet,” approximate the consistency of endogenous tears, aid in natural tear build-up, or otherwise provide temporary relief of dry eye symptoms and conditions upon ocular administration the eye are known in the art. Such compounds may enhance the viscosity of the formulation, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P. Other compounds may also be added to the ophthalmic formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Formulations formulated for the treatment of dry eye-type diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous formulations which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration; (ii) are safe; and (iii) provide the appropriate delivery vehicle for the topical administration of an effective amount of (11β,17α),-17-[(Ethoxycarbonyl)oxy]-11-hydroxy-3-oxoandrosta-1,4-diene-17-carboxylic acid chloromethyl ester. Examples of artificial tears formulations useful as artificial tears carriers include, but are not limited to, commercial products, such as Moisture Eyes™ Lubricant Eye Drops/Artificial Tears, Moisture Eyes™ Liquid Gel lubricant eye drops, Moisture Eyes™ Preservative Free Lubricant Eye Drops/Artificial Tears and Moisture Eyes™ Liquid Gel Preservative Free Lubricant Eye Drops/Artificial Tears (Bausch & Lomb Incorporated, Rochester, N.Y.). Examples of phospholipid carrier formulations include those disclosed in U.S. Pat. Nos. 4,804,539 (Guo et al.), U.S. Pat. No. 4,883,658 (Holly), U.S. Pat. No. 4,914,088 (Glonek), U.S. Pat. No. 5,075,104 (Gressel et al.), U.S. Pat. No. 5,278,151 (Korb et al.), U.S. Pat. No. 5,294,607 (Glonek et al.), U.S. Pat. No. 5,371,108 (Korb et al.), U.S. Pat. No. 5,578,586 (Glonek et al.), the contents of each of which are incorporated by reference herein.

The preferred formulations of the present invention are intended for administration to a human patient suffering from ophthalmic diseases such as dry eye or symptoms of dry eye. Preferably, such formulations will be administered topically. In general, the doses used for the above described purposes will vary, but will be in an effective amount to eliminate or improve dry eye conditions. Generally, 1-2 drops of such formulations will be administered from once to many times per day. The formulation is intended to be provided as a package for the treatment of dry eye, the package would include the pharmaceutical formulation comprising Loteprednol etabonate contained in a pharmaceutically acceptable container; a written package insert containing instructions for using the formulation for the treatment of dry eye; and outer packaging identifying the pharmaceutical formulation contained therein. In certain embodiments wherein the formulation is preservative free, the package would contain a pharmaceutically acceptable container suitable for single use by a user of the packaged formulation. In such embodiments it is envisioned that the outer packaging would contain at least one pharmaceutically acceptable container containing the Loteprednol etabonate formulation. Preferably the outer packing would contain a multiplicity of single use containers, for example, enough single use containers to provide for a one-month supply of the formulation.

According to one embodiment of the present invention, there is now a method of producing a sterile Loteprednol etabonate gel that is characterized in that a sterile polyacrylate gel is produced, but that Loteprednol etabonate is separately sterilized and incorporated into the acrylate gel, in a suitable amount, under aseptic conditions. Alternatively, the sterile Loteprednol etabonate is suspended with a part of the solution, which may contain a sterile tonicity agent, used for the production of the polyacrylate gel, and this suspension is then homogenously mixed in with the separately sterilized polyacrylate gel.

It has been shown that a sterile Loteprednol etabonate gel in a polyacrylate base can be satisfactorily produced when certain method steps are followed in its production. According to one embodiment of the present invention, an aqueous polyacrylate suspension is made and then autoclaved under sterile conditions. This acrylate suspension is mixed with a sterile-filtrated solution of preserving agent, isotonicity agent, and chelating agent. After careful and thorough mixing of the starting materials, the addition of sterile-filtrated caustic soda solution initiates gel formation, and the gel is further subjected to agitation until it is homogenous. Meanwhile the Loteprednol etabonate or its pharmaceutically acceptable ester is sterilized. This can be accomplished by dissolving the active substance in a suitable amount of solvent, for example ethyl acetate, subjecting the solution to sterile filtration, and precipitating the active substance, for example, through the addition of sterile water with an anti-microbial agent under aseptic conditions. The microbially sterile Loteprednol etabonate or its pharmaceutically acceptable ester is then triturated or ground to a powder with about three to ten times that amount of the gel base. The remaining amount of gel is then incorporated in the concentrate by thorough mixing. The finished gel preparation is then conventionally decanted or drawn off under sterile conditions. In an alternative variation of this method, the microbially sterile Loteprednol etabonate or its pharmaceutically acceptable ester can be, to a large extent, suspended in a part of the aqueous solution of the tonicity agent. The polyacrylate gel can be made in a conventional manner with the remaining amount of isotonic agent and separately the isotonic suspension of the Loteprednol etabonate can be homogenously mixed with the polyacrylate under sterile conditions.

This sterile gel is well acceptable to the patient, because its application does not have the disadvantage of known ointments and is not oily. Stability has been proven, so that the gel has a relatively long shelf life without any change in its physical properties. In particular, there is no crystal growth of the active ingredient. Such a sterile gel preparation represents a significantly improved form of application in the ophthalmological field. The present invention will be further explained and illustrated by the Example that follows.

The invention will now be further described by way of several examples that are intended to describe but not limit the scope of the invention as defined by the claims herein.

Representative eye drop formulations are provided in Examples 1-3 below.

### EXAMPLE 1

| Ingredient | Amount |
|---|---|
| Phase I | |
| Carbopol 934P NF (Acrylic acid-based polymer) | 0.25 gm |
| Purified Water | 99.75 gm |
| Phase II | |
| Propylene Glycol | 5.0 gm |
| EDTA | 0.1 mg |
| Loteprednol Etabonate | 50.0 gm |

Mix five parts of phase II with twenty parts of phase I for more than 15 minute and adjust pH to 6.2-6.4 using 1 N NaOH.

### EXAMPLE 2

| Ingredient | Amount |
|---|---|
| Phase I | |
| Carbopol 934P NF (Acrylic acid-based polymer) | 0.25 gm |
| Purified Water | 99.75 gm |
| Phase II | |
| Propylene Glycol | 3.0 gm |
| Triacetin | 7.0 gm |
| Loteprednol Etabonate | 50.0 gm |
| EDTA | 0.1 gm |

Mix five parts of phase II with twenty parts of phase I for more than 15 minutes and adjust pH to 6.2-6.4 using 1 N NaOH.

### EXAMPLE 3

| Ingredient | Amount |
|---|---|
| Phase I | |
| Carbopol 934P NF (Acrylic acid-based polymer) | 0.25 gm |
| Purified Water | 99.75 gm |
| Phase II | |
| Propylene Glycol | 7.0 gm |
| Glycerin | 3.0 mg |
| Loteprednol Etabonate | 50.0 gm |
| EDTA | 0.1 mg |
| BAK | 01-0.2 mg |

Mix five parts of phase II with twenty parts of phase I for more than 15 minutes and adjust pH to 6.2-6.4 using 1 N NaOH.

### EXAMPLE 4

This prophetic example illustrates a method of making a gel according to the present invention, although the production of larger amounts of gel may be necessary to meet commercial demands. In the present example, the gel is produced with water that is suitable for injection purposes (injection grade). To produce 500 g of polyacrylate gel, 1.220 g of polyacrylic acid (packaged under the trademark “Carbopol 980 NF”) is carefully suspended, with the aid of an ultrasonic apparatus, in about 700 ml water and autoclaved for 20 minutes at 121° C. and 2 bar pressure. In 700 ml of sterile injection-grade water is then dissolved 0.050 g of benzalkonium chloride (BAK), 20.000 g sorbitol and 0.050 g of sodium EDTA (X 2H₂O), which is then subjected to sterile filtering (Sartorius®. Cellulose nitrate filter, order no. 11307-50ACN, 0.2 μm) into a sterile vessel. The sterile-filtered salt solution is then mixed, with strong agitation, into the autoclaved polyacrylate suspension. Sterile water in the amount of 1958.121 g is then added, and the solution is subjected to further agitation for 5 to 10 minute. Subsequently, strong sodium hydroxide in the amount of 0.465 g is dissolved in exactly 40 g of injection-grade water. This caustic soda is then introduced drop-wise under agitation over a sterile filter (Millex-GS, 0.22 μm, SLGS 025 BS der Fa. Millipore). The mixture is agitated until the formation of a completely homogenous gel.

A microbially sterile Loteprednol etabonate in the amount of 5 g is then slowly and carefully mixed with about 30 to 50 g of the gel. The gel is subjected to sterile filtration of the solution, and separation with water containing a bacteriocide under sterile conditions. After the Loteprednol etabonate is accordingly dissolved in the given amount of gel, the rest of the gel, in total 495 g, is carefully incorporated into the initial material. All method steps are carried out under aseptic conditions.

The prepared gel is likewise drawn off in tubes under aseptic conditions. By an alternative method, the microbially sterile Loteprednol etabonate is suspended in a sterile-filtrated isotonic solution of 700 ml water, 0.050 g benzalkonium chloride, 20.000 g sorbitol and 0.050 g of disodium EDTA. This solution is then, as already described, incorporated, under strong agitation, in the autoclaved polyacrylate suspension. Further adaptation or modification of the invention, corresponding to the described production of sterile polyacrylate gel, falling within the scope of the following claims may occur to the skilled artisan.

This invention has been described by reference to certain preferred embodiments; however, it should be understood that it may be embodied in other specific forms or variations thereof without departing from its special or essential characteristics. The embodiments described above are therefore considered to be illustrative in all respects and not restrictive, the scope of the invention being indicated by the appended claims rather than by the foregoing description.

The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

What is claimed is:

1. A method of producing a sterile Loteprednol etabonate gel characterized in that an aqueous suspension comprising a polyacrylic acid or polyacrylate is produced and converted into a sterile polyacrylate gel, Loteprednol etabonate or its pharmaceutically acceptable ester is separately sterilized and then incorporated, under aseptic conditions, into a corresponding amount of either the sterile polyacrylate gel or the aqueous suspension comprising the polyacrylic acid or polyacrylate polymer, which aqueous suspension is then transformed into a gel.

2. The method of claim 1 characterized in that the sterile polyacrylate gel is produced by autoclaving an aqueous suspension comprising a polyacrylic acid or polyacrylate and adding an effective amount of a sterile caustic soda.

3. The method of claim 2 characterized in that the aqueous suspension comprising a polyacrylic acid or polyacrylate A pharmaceutically acceptable composition comprising acrylic anionic polymers such a hyaluronic acid, alginates, carboxy methyl cellulose, water, osmotic agent such as propylene glycol, glycerin, sugars, mannitol, amino acid, chelating agent such as EDTA, DEQUEST, and any pharmaceutically active ingredient or combination of pharmaceutically active ingredients is mixed with an aqueous solution of a pharmaceutically acceptable preservative.

4. The method of claim characterized in that the aqueous suspension comprising a polyacrylic acid or polyacrylate is mixed with a sterile solution of a complexing agent.

5. The method according to claim characterized in that the aqueous suspension of a polyacrylic acid or polyacrylate is mixed with a sterile aqueous solution of an isotonicity agent.

6. The method according to claim 2 characterized in that the Loteprednol etabonate is dissolved in a solvent for Loteprednol etabonate, subjected to sterile filtration, separated out, and microbially sterilized under sterile conditions.

7. The method according to claim 2 characterized in that the desired total amount of Loteprednol etabonate or its pharmaceutically acceptable ester is triturated, under aseptic conditions, with about 1/10 of the total amount of polyacrylate and then incorporated in a homogenous mixture with the rest of the gel.

8. A method according to claim 2 for the production of a sterile Loteprednol etabonate gel characterized in that the sterile Loteprednol etabonate or its pharmaceutically acceptable ester is suspended with a sterile solution of a tonicity agent, preservative, and complexing agent, and then mixed with a sterile suspension of the polyacrylic acid or polyacrylate, which is then converted into a gel.

9. A method according to claim 3 characterized in that the aqueous suspension comprising a polyacrylic acid or polyacrylate is mixed with an aqueous solution of benzalkonium chloride.

10. The method of claim 4 characterized in that the aqueous suspension comprising a polyacrylic acid or polyacrylate is mixed with a sterile solution of EDTA or its pharmaceutically acceptable salt.

11. The method according to claim 5 characterized in that the aqueous suspension of a polyacrylic acid or polyacrylate polymer is mixed with a sterile aqueous solution of sorbitol.

12. A method of producing sterile Loteprednol etabonate gel characterized in that an aqueous suspension comprising a polyacrylate acid or polyacrylate is produced and converted into a sterile polyacrylate gel, Loteprednol etabonate or its pharmaceutically acceptable ester is separately sterilized and then incorporated, under aseptic conditions, into a corresponding amount of either the sterile polyacrylate gel or the aqueous suspension comprising the polyacrylic acid or polyacrylate polymer, which aqueous suspension is then transformed into a gel, wherein the sterile polyacrylate gel is produced by autoclaving and the Loteprednol etabonate or its pharmaceutical acceptable ester is dissolved in a solvent for Loteprednol etabonate and subjected to sterile filtration prior to being incorporated into the sterile polyacrylate gel or into the aqueous suspension comprising the polyacrylate acid or polyacrylate polymer that is subsequently transformed into a gel.

13. A pharmaceutically acceptable composition comprising acrylic acid-based polymer, water, propylene glycol, EDTA and Loteprednol etabonate.

14. The pharmaceutically acceptable composition claim 13 wherein the composition further compromises triacetin.

15. The pharmaceutically acceptable composition of claim 13 wherein the formulation compromises acrylic acid-based polymer, water, propylene glycol, glycerin, EDTA, benzalkonium chloride and Loteprednol etabonate.

16. A pharmaceutically acceptable composition comprising:
at least one anionic polymer;
water;
at least one osmotic agent; and,
a pharmaceutically active ingredient or combination of pharmaceutically active ingredients.
